(19) United States
(12) Patent Application Publication
(10) Pub. No.: US 2021/0338178 A1
(43) Pub. Date: Nov. 4, 2021

(54) ADJUSTABLE PRIVACY SCREEN

(71) Applicant: LANKENAU INSTITUTE FOR MEDICAL RESEARCH, Wynnewood, PA (US)

(72) Inventor: Jeshahnton Essex, Glen Mills, PA (US)

(21) Appl. No.: 17/245,033

(22) Filed: Apr. 30, 2021

Related U.S. Application Data
(60) Provisional application No. 63/018,945, filed on May 1, 2020.

(51) Int. Cl. A61B 5/00; A47H 1/022; A47H 13/01; A47H 13/04; A47H 99/00

(52) U.S. Cl. CPC A61B 5/746; A47H 1/022; A61B 5/1117; A47H 13/04; A47H 99/00; A47H 13/01

(57) ABSTRACT

An adjustable privacy screen has a length-adjustable support member that can be wedged between adjacent walls. The support member may include locking telescoping tubes, and optionally, semi-rigid brace members that may be bent to wedge the device between adjacent walls. An opaque privacy drape is releasably attachable, e.g., by adhesive or mechanical fasteners, to the support member. A sensor mounted on the support member is operable to cause a signaling device on the support member to provide an alert signal to a caregiver upon detection of a predetermined condition by the sensor. The sensor may also be operable to cause a mechanism to operate to release the privacy screen from its position wedged between adjacent walls. One or more legs may be releasably attachable to the support member.

### CROSS-REFERENCE TO RELATED APPLICATION

This application claims the benefit of priority, under 35 U.S.C. § 119(e), of U.S. Provisional Patent Application No. 63/018,945, filed May 1, 2020, the entire disclosure of which is hereby incorporated herein by reference.

### FIELD OF THE INVENTION

The present invention relates generally to protecting persons from bodily injuries relating to falls, and more particularly, to a device providing a privacy screen for use during toileting activities by a person being monitored for fall prevention purposes.

### DISCUSSION OF RELATED ART

Falling is a significant cause of mortality or injury among patients recovering from surgery, the elderly and the infirm, especially within a hospital or a caregiving facility. Injuries and death due to falls are an issue that every hospital in the world faces. In-patient falls cost US health systems over $34 billion annually. It has been estimated that 1 million falls occur in North American hospitals annually. With implementation of the Centers for Medicare and Medicaid Service No-Pay Policy for expenses related to hospital-acquired falls, hospitals are responsible for all fall-related costs.

Studies have shown that falls and the resulting injuries very frequently occur in the bathroom during toileting activities. Accordingly, some hospitals or other care facilities place patients on a fall precaution watch based on clinical or other findings. The fall precaution watch may result in implementation of a protocol or procedures intended to increase the likelihood that a patient fall will be avoided. For example, the fall precaution watch may involve implementation of a procedure by which a patient is instructed to alert a nurse/caregiver before rising from bed and/or engaging in toileting activities, and the nurse/caregiver is obligated to accompany the patient at all times while ambulating to the bathroom, and to join the patient in the bathroom during toileting activities so that the patient can be very closely monitored so that the nurse/caregiver can provide immediate assistance in the event of a loss of balance etc. to avoid a patient fall and injury.

While this approach may be helpful, there may be compliance issues, in that the patient may avoid alerting the nurse/caregiver because the patient does not want to suffer the corresponding loss of privacy of having a nurse/caregiver join the patient in the bathroom during toileting activities. Further, the patient may not want the nurse/caregiver to remain in close proximity to the patient during toileting activities, even if the nurse/caregiver is present in the bathroom, to retain a measure of privacy. Accordingly, implementation of a fall precaution policy, or application of fall precautions to a particular patient, may not be as effective as desired at avoiding patient falls and resulting injuries.

What is needed is a device that provides for enhanced patient privacy while being supervised during toileting activities, so that fall precaution policies are more closely followed and/or are more effective in avoiding patient falls and resulting injuries.

### SUMMARY

The present invention provides an adjustable privacy screen device that provides for enhanced patient privacy while being supervised during toileting activities, so that fall precaution policies are more closely followed and/or are more effective in avoiding patient falls and resulting injuries. In one embodiment, the adjustable privacy screen comprises: an elongated support member having a first end and a second end opposite said first end, said elongated support member being configurable to vary a length between said first end and said second end; and a drape releasably attachable to said support member. The adjustable privacy screen may include an alarm system comprising: a sensor mounted on said support member; a signaling device; and a controller mounted on said support member and operatively connected to the sensor to cause said signaling device to provide an alert signal to the caregiver upon detection of a predetermined condition by the sensor.

### BRIEF DESCRIPTION OF THE FIGURES

An understanding of the following description will be facilitated by reference to the attached drawings, in which:

FIG. 1 is a top view of an adjustable privacy screen in accordance with an exemplary embodiment of the present invention;

FIG. 2 is a side view of the adjustable privacy screen of FIG. 1, shown supporting a privacy drape;

FIG. 3 is a perspective of the adjustable privacy screen of FIG. 1, shown with supportive legs attached; and

FIG. 4 is a perspective view of the adjustable privacy screen of FIG. 1, shown for illustrative purposes in an operative position in an exemplary bathroom.

### DETAILED DESCRIPTION

The present invention provides a privacy screen that provides for enhanced patient privacy while being supervised during toileting activities, so that fall precaution policies are more closely followed and/or are more effective in avoiding patient falls and resulting injuries. The privacy screen is adjustable for use in a wide variety of bathroom environments of different dimensions. Further, the privacy screen is configured to provide privacy by acting as a support for a privacy drape while also providing the patient ease of passage so as not to act as a restraint upon patient ambulation during egress from a bathroom. In certain embodiments, the privacy screen includes a sensor and an alarm for issuing an alert signal upon detection of touch and/or movement by the patient, so as to alert a nurse/caregiver that the patient is beginning to ambulate and/or exit the bathroom after toileting activities without assistance, e.g., if the event that the attending nurse/caregiver is distracted or moves out of a line of sight with the patient.

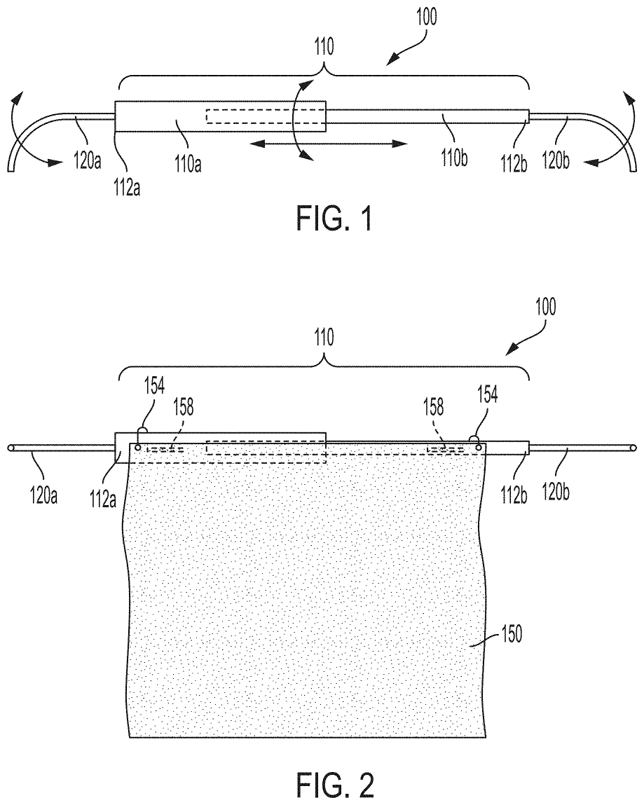

Referring now to the exemplary and illustrative non-limiting embodiment shown in the drawings, an adjustable privacy screen 100 is shown in accordance with an exemplary embodiment of the present invention. As will be appreciated from FIGS. 1 and 2, the exemplary privacy screen 100 includes a rigid support member 110 and a pair of semi-rigid brace members 120. In combination, the rigid support member 110 and the flexible semi-rigid brace members 120a, 120b provide structure for effectively wedging the privacy screen between adjacent walls of a bathroom in a friction fit, so as to support the privacy screen 100 therebetween, and for supporting a privacy drape serving to block at least a portion of the patient's body from an attending nurse/caregiver's view during toileting (or other) activities, as discussed in greater detail below.

The privacy screen 100 is length-adjustable so as to span a range of widths of bathroom environments in which the privacy screen may be used. In part, the exemplary privacy screen 100 is length-adjustable in that the rigid support member 110 is length-adjustable. In this embodiment, the rigid support member 110 is constructed as a pair of nested telescoping tubes 110a, 110b configured such that they may be adjusted in position relative to one another and then be locked into selected relative positions. Any suitable structures may be used to provide this functionality. In the exemplary embodiment, the rigid support member 110 is structured and operable similarly to a conventional adjustable shower rod, so that the tubes can be rotated about their axis of elongation to unlock the tubes 110a, 110b, at which point one tube may be translated along the axis of elongation relative to the other, and then the tubes can be counter-rotated about their axis of elongation to interlock the tubes and fix them into relative positions. In this manner, the length of the support member can be increased or decreased to correspond to the width of an opening between adjacent walls of a bathroom between which the privacy screen is intended to be installed.

In the embodiment, the privacy screen 100 is also length-adjustable due in part to the flexibility of the semi-rigid brace members 120a, 120b. Each semi-rigid brace member 120a, 120b extends from a respective end 112a, 112b of the tubes of the rigid support member 110. Each semi-rigid brace member 120a, 120b, is a reasonably stiff, but readily manually, bendable member, such manual force associated with inserting the privacy screen 100 between adjacent walls causes the semi-rigid brace members 120a, 120b to bend, and then tend to resile, to effectively wedge the privacy screen 100 between the adjacent walls. Notably, this type of construction provides sufficient friction with the walls to support the privacy screen 100 (including any privacy drape) between the walls, while also avoiding a situation in which the privacy screen is fixedly attached to the walls in a manner that could serve as a restraint impeding a patient from passing by the privacy screen or existing the bathroom, because the privacy screen may be easily dislodged from between the walls by simple application of manual force by hand, waist, hip, leg, etc., without a need to apply the force in any particular direction or manner. The semi-rigid brace members 120a, 120b may have any suitable construction. In the exemplary embodiment, each semi-rigid brace member 120a, 120b is constructed to include a semi-rigid, e.g., braided, steel stranded, wire that provides the rigidity, flexibility and tendency to resile when bent, and a rubber or other relatively high-friction coating or covering on the wire, to promote friction between the semi-rigid brace member 120a, 120b and the wall of the bathroom, which may be a fairly low-friction surface, such as ceramic tile. FIG. 1 shows a top view of the privacy screen 100, and shows the semi-rigid brace members 120a, 120b in a partially-bent position for illustrative purposes. In one embodiment, the semi-rigid brace members 120a, 120b are straight in their unbent (relaxed) state. In another embodiment, the semi-rigid brace members 120a, 120b are curved in their unbent (relaxed) state, which can facilitate wedging of the privacy screen between adjacent walls.

Preferably, the support member 110 and any semi-rigid support members 120a, 120b are constructed of materials that can be easily cleaned for disinfection purposes, so that the support member 110 can be reused for a single patient or across multiple patients without significant cross-contamination concerns. Various materials are suitable for this purpose, as will be appreciated by those skilled in the art.

FIG. 2 is a side view of the adjustable privacy screen 100 of FIG. 1, shown supporting a privacy drape 150. The privacy drape 150 is constructed of an opaque material, so as to act as a visual privacy screen obscuring at least a portion of the patient from view (e.g., the torso or mid-section of the patient), while leaving a portion of the patient viewable above the privacy drape 150 (e.g., the head and shoulders of the patient) during toileting activities. In this manner, the privacy drape 150, and thus the privacy screen 100, provides a measure of privacy to a patient by obscuring a portion of the patient from the view of the attending nurse/caregiver. The privacy drape 150 is preferably constructed of lightweight and inexpensive material, such as a thin plastic or paper sheet, so as to promote disposal of the privacy drape after each use of the privacy screen for toileting activities. The privacy screen and/or privacy drape are configured such that the privacy screen is readily and releasably detachable from the support member 110 and/or semi-rigid brace members 120a, 120b. For example, the support member 110 may include clamps or clips for clasping the privacy drape 150, or the support members 110 may have hooks, pins or pegs 154 for hanging the privacy drape 150 from the support member. Alternatively, the privacy drape 150 may include hooks for suspending the privacy drape 150 from the support member 100. By way of alternative example, the privacy drape 150 may include fields 158 of adhesive, such as pressure-sensitive adhesive, for temporarily and removably adhering the privacy drape 150 to the support member 100. Any suitable structures or methods may be used for attaching the privacy drape 150, as will be appreciated by those skilled in the art.

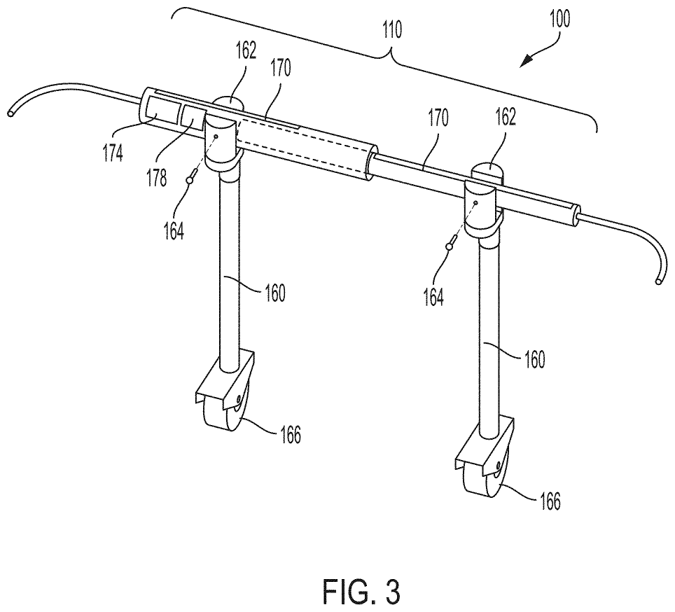

Optionally, the adjustable privacy screen 100 may be used in conjunction with supportive legs 160, as shown in FIG. 3, which omits the privacy drape of FIG. 2 for illustrative clarity. As shown in FIG. 3, a pair or upwardly extending legs 160 may be provided for attachment to the support member 110. The legs and/or support member 110 may be configured for interlocking attachment with the support member 110. For example, the legs 160 may terminate at their upper ends with brackets 162 configured to receive a pin 164 passed through or over the support member to interlock the legs with the support member 110. Optionally, the legs 160 may include rotatable wheels 166 at their lower ends to facilitate rolling movement of the privacy screen 100 along the floor. Preferably, the legs are releasably attached, so that they may be readily removed for compact storage purposes, e.g., in a patient's bathroom. The legs can facilitate support of the support member 110, can facilitate installation of the support member 110 at an appropriate or predetermined height, and can facilitate the patient is pushing the privacy screen out of the patient's path when exiting the bathroom, so that the privacy screen 100 does not act as a restraint or patient ambulation or egress from the bathroom.

In certain embodiments, the privacy screen 100 further includes an alarm system for alerting an attending nurse/caregiver that the patient is ready to or beginning to ambulate and/or to exit the bathroom or pass by the privacy screen. Accordingly, in such embodiments, the privacy screen 110 further includes a sensor 170 mounted on the support member 110, a signaling device 174 mounted on the support member 110 and operable to provide an audible and/or visible signal to an attending nurse/caregiver, such as an audible alarm sound, and a controller 178 mounted on or in the support member 100 and operatively connected to the sensor 170 and the signaling device 174, and operable to detect touch and/or pressure on the sensor 170 by the patient and responsively activate the signaling device 174 to provide an alert signal to the attending nurse/caregiver in the event of a detected touch/pressure by the sensor 170. Any suitable hardware and/or software may be incorporated to provide this alarm system functionality.

In certain embodiments, the privacy screen 100 is further configured to collapse after a predetermined time, and/or in response to applied pressure to the privacy screen/support member 110 itself. In this embodiment, the support member 110 is configured to operate very similar to “crash bars” used in sensitive areas of a hospital such as the ED and Maternity Units. More particularly, the privacy screen 100 includes an audible alarm and sensor that is configured to sense applied pressure/weight. The sensor is mounted on and/or spans the support member 110, which would always be positioned directly in front of the patient during normal use. When the nurse/caregiver assists the patient to the toilet area, he/she can activate the sensor by placing the privacy bar and drape upright and in position on the walls. This automatically configures the sensor so that it is not dependent upon the caregiver remembering to manually activate the sensor or not. When pressure/weight is applied to the support member 110 (e.g., as a patient prepares or attempts to exist the toilet area), the alarm activates and after a few (e.g., 10-15) seconds, the support member 110 and drape collapses by way of a mechanism on the support member that ceases to outwardly bias the inner support member 110b (or at least one of the semi-rigid brace members 120a, 120b), to allow it to move inwardly. The sensor/alarm may be powered by batteries that will include a low battery function to let the end user know when the batteries should be replaced.

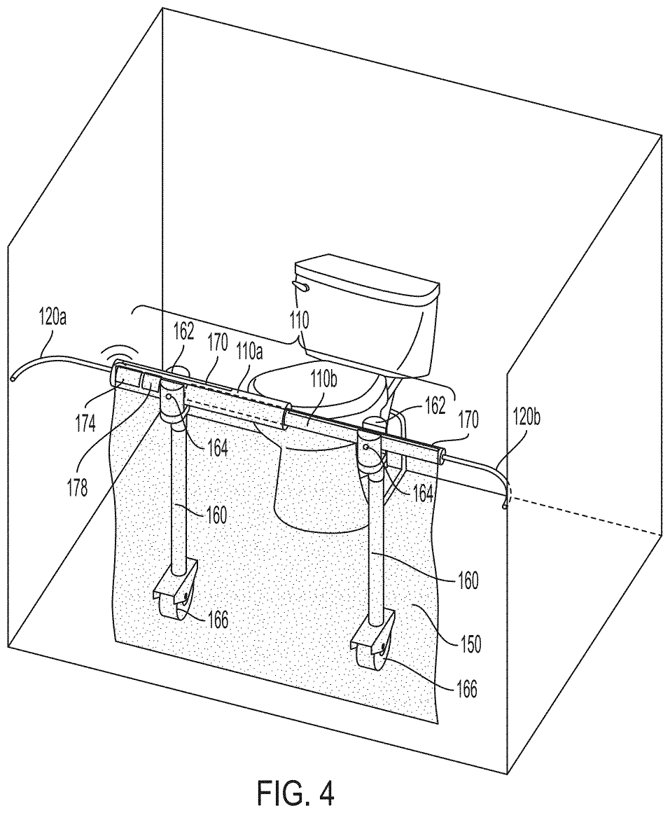

By way of illustrative example, the adjustable privacy screen 100 may be used as follows. When the privacy screen 100 is intended to be used in a particular bathroom for a particular instance of patient toileting, the nurse caregiver may first rotate one or both of the telescoping tubes 110a, 110b about their axis of elongation, so as to uncouple/unlock the relative positions of the tubes 110a, 110b. The caregiver may then apply a coarse adjustment of the length of the support member 110 by translating one or both of the tubes 110a, 110b relative to each other until the overall combined length of the support member 110 and the semi-rigid brace members 120a, 120b is slightly greater than a distance between adjacent walls where the privacy screen 100 is to be installed, at which point one or both of the tubes 110a, 110 may be counter-rotated to lock the tubes into position and fix the left of the privacy screen 100. If desired, legs 160 may be attached to the support member 110. The privacy screen 100 may then be pressed into place between the walls, as shown in FIG. 4, causing the semi-rigid members to be partially bent/deflected by engagement with the walls in the process, thereby forming a tight friction fit between the support member 110/semi-rigid brace members 120a, 120b, and the walls, to fix the privacy screen into place. The privacy drape 150 may then be attached to the support member 110, at which point the privacy screen 100 is ready for use to act as a visual privacy screen to partially obscure the patient from view of an attending nurse/caregiver during use of the bathroom for toileting activities.

In the event that the nurse/caregiver is momentarily distracted or otherwise inattentive, action by the patient in an attempt to pass by or remove the privacy screen will likely result in contact of the patient with a sensor 170 of the alarm system. In this event, the controller 178 will detect the patient's contact with the sensor 170, and the controller will responsively activate the signaling device 174 to cause an audible, visual or other alert for capturing the attention of the nurse/caregiver so that attention can be returned to the privacy screen 100 and patient, so that patient can be closely monitored and/or assisted, and a fall can be avoided. In the event that the nurse/caregiver does not return attention to the privacy screen 100 and patient, the patient's attempts to move and/or pass by the privacy screen 100 may in certain embodiments result in automated dislodging and/or collapsing of the privacy screen from its wedged position between the walls (e.g., by operation of the mechanism, under control of the controller 178, to cease to outwardly bias the inner support member 110b and/or at least one of the semi-rigid brace members 120a, 120b, to allow it to move inwardly, and to allow the patient to pass by freely, so as not to serve as a restraint to patient ambulation or egress from the bathroom.

In certain embodiments, the disposable drapes are provided with “odor guard” technology (e.g., chemical treatment to provide a fragrance and/or to neutralize odor) similar to that used with many conventional trash bags.

In certain embodiments, the privacy screen may include or communicate with appropriate hardware and/or software tracking how many times the sensor was activated, how many times support member collapse occurred, whether the sensor/alarm has been activated, and/or other pertinent details that will provide reporting useful for the caregiver, end user and/or supervisory team.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. An adjustable privacy screen comprising:
an elongated support member having a first end and a second end opposite said first end, said elongated support member being configurable to vary a length between said first end and said second end; and
a drape releasably attachable to said support member.

2. The adjustable privacy screen of claim 1, wherein said elongated support member comprises first and second members adjustable and securable in a plurality of different longitudinal positions relative to one another.

3. The adjustable privacy screen of claim 2, wherein said first and second members are arranged in a telescoping tube arrangement.

4. The adjustable privacy screen of claim 1, wherein said elongated support member comprises at least one semi-rigid brace member that defines at least one of said first end and said second end, wherein said at least one semi-rigid brace member is readily bendable to vary the length between said first end and said second end.

5. The adjustable privacy screen of claim 1, wherein said elongated support member comprises a pair of semi-rigid brace members, each of said pair of semi-rigid brace members defining a respective one of said first end and said second end, wherein each of said pair of semi-rigid brace members is readily bendable to vary the length between said first end and said second end.

6. The adjustable privacy screen of claim 1, wherein said support member comprises at least one of a clamp, a clip, a hook, a pin and a peg for hanging said drape to releasably attach it to said support member

7. The adjustable privacy screen of claim 1, wherein said support member comprises at least one field of adhesive configured to temporarily and removably adhering said privacy drape to said support member.

8. The adjustable privacy screen of claim 1, wherein said drape comprises one of a hook, a pin and a peg for hanging the drape to releasably attach it to said support member.

9. The adjustable privacy screen of claim 1, further comprising at least one leg attached to said elongated support member and extending transversely thereto.

10. The adjustable privacy screen of claim 9, wherein said leg comprises a bracket dimensioned to receive said support member.

11. The adjustable privacy screen of claim 10, wherein at least one of said leg and said support member defines an opening dimensioned to receive a locking pin, said locking pin being insertable into said opening to retain said support member within said bracket.

12. The adjustable privacy screen of claim 10, each of said at least one leg comprises a rotatable wheel at its lower end.

13. The adjustable privacy screen of claim 1, further comprising an alarm system for alerting a caregiver, said alarm system comprising:
a sensor mounted on said support member;
a signaling device; and
a controller mounted on said support member and operatively connected to the sensor to cause said signaling device to provide an alert signal to the caregiver upon detection of a predetermined condition by the sensor.

14. The adjustable privacy screen of claim 13, wherein said sensor is configured to detect one of pressure and a touch by a person, and wherein.

15. The adjustable privacy screen of claim 13, further comprising a mechanism on the support member operable to cease outward biasing of at least a portion of said support member.

16. An adjustable privacy screen comprising:
an elongated support member comprising:
a first tube positioned within and selectively lockable to a second tube in a telescoping arrangement; and
a first semi-rigid brace member joined to one of said first tube and said second tube;
said elongated support member having a first end and a second end opposite said first end, said elongated support member being configurable to vary a length between said first end and said second end; and
a drape releasably attachable to said support member.

17. The adjustable privacy screen of claim 16, wherein said elongated support member further comprises a second semi-rigid brace member joined to another of said first tube and said second tube.

18. The adjustable privacy screen of claim 16, wherein said support member comprises one of a clamp, a clip, a hook, a pin, and a pen for handing said drape to releasably attach it to said support member.

19. The adjustable privacy screen of claim 16, wherein said support member comprises at least one field of adhesive configured to temporarily and removably adhering said privacy drape to said support member.

20. The adjustable privacy screen of claim 16, wherein said drape comprises one of a hook, a pin and a peg for hanging the drape to releasably attach it to said support member.

21. The adjustable privacy screen of claim 16, further comprising at least one leg attached to said elongated support member and extending transversely thereto.

22. The adjustable privacy screen of claim 21, wherein said leg comprises a bracket dimensioned to receive said support member.

23. The adjustable privacy screen of claim 16, further comprising an alarm system for alerting a caregiver, said alarm system comprising:
a sensor mounted on said support member;
a signaling device; and
a controller mounted on said support member and operatively connected to the sensor to cause said signaling device to provide an alert signal to the caregiver upon detection of a predetermined condition by the sensor.

24. An adjustable privacy screen comprising:
an elongated support member having a first end and a second end opposite said first end, said elongated support member being configurable to vary a length between said first end and said second end;
a drape releasably attachable to said support member; and
an alarm system for alerting a caregiver, said alarm system comprising:
a sensor mounted on said support member;
a signaling device; and
a controller mounted on the support member and operatively connected to the sensor to cause said signaling device to provide an alert signal to the caregiver upon detection of a predetermined condition by the sensor.

25. The adjustable privacy screen of claim 24, further comprising at least one leg removably attached to said elongated support member and extending transversely thereto.

26. The adjustable privacy screen of claim 25, wherein said leg comprises a bracket dimensioned to receive said support member.

27. The adjustable privacy screen of claim 24, wherein said support member comprises:
a first tube positioned within and selectively lockable to a second tube in a telescoping arrangement.

28. The adjustable privacy screen of claim 27, wherein said support member further comprises:
at least one semi-rigid brace member joined to one of said first tube and said second tube.
